(19) United States
(12) Patent Application Publication
(10) Pub. No.: US 2026/0224334 A1
(43) Pub. Date: Aug. 6, 2026

(54) Inorganic-Protein Coating on Mg-Based Biomaterials

(71) Applicants: Hongbo ZENG, Edmonton (CA); Ziqian Zhao, Edmonton (CA)

(72) Inventors: Hongbo ZENG, Edmonton (CA); Ziqian Zhao, Edmonton (CA)

(21) Appl. No.: 19/454,932

(22) Filed: Jan. 21, 2026

Related U.S. Application Data
(60) Provisional application No. 63/747,427, filed on Jan. 21, 2025.

(51) Int. Cl. A61C 8/00; A61C 8/02; A61L 27/04; A61L 27/34

(52) U.S. Cl. CPC A61C 8/0013; A61C 8/0006; A61L 27/047; A61L 27/34; A61L 2430/12

(57) ABSTRACT

A composite coating is formed from NaMgF₃ nanoparticles and a protein on an magnesium-based biomaterial, by immersing the biomaterial in a solution of the NaF and the protein. The composite coated biomaterial may be used as a surgical implant, such as in dental implant surgery.

### FIELD

This disclosure relates to a coating comprising an inorganic component and a protein component, particularly for use with magnesium-based biomaterials.

### BACKGROUND

Dental and orthopedic biomaterials, such as fixation plates for broken bone healing and barrier membranes for dental implantation are widely used and are commercially valuable.^{[1][2]} However, such biomaterials, which include stainless steel, titanium alloys, ultrahigh molecular weight polyethylene, and polycaprolactone polymers, have shown increasing limitations. Hard metallic biomaterials can induce stress-shielding effects, leading to peri-implant bone loss and necessitating additional removal surgery. Soft polymeric biomaterials cannot function as load-bearing replacements and also raise clinical toxicity concerns when degraded.

Magnesium-based biomaterials can overcome such limitations and have been investigated for decades due to their bone-like mechanical properties and biodegradability^{[3]}. However, Mg-based biomaterials are susceptible to rapid bio-corrosion.

Bio-corrosion or bio-degradation avoids the secondary surgery for implant removal, and Mg ions released from Mg-based biomaterial degradation promote bone regeneration and accelerate new bone tissue formation.^{[2, 4]} These benefits of rapid degradation induced by bio-corrosion are offset by structural integrity deterioration, reduced mechanical strength, and side effects of health risks like subcutaneous emphysema. Thus, it is desirable to provide protective coatings to mitigate dysfunction.

Constructing an anticorrosion coating appears to be more feasible and safer than traditional microalloying strategies. Microalloying generally involve accelerated degradation and toxic elements.^{[3b, 3c, 3f, 5]} However, directly constructing a coating on Mg substrates is challenging due to the high reactivity of Mg surfaces, which causes surface alkalinization (pH ~10.5) to degrade the coatings and generates numerous H₂ bubbles, resulting in porous coatings or coating delamination.^{[4a, 6]}

Traditional used organic coatings are highly compact but suffer from insufficient mechanical durability and low interfacial adhesion to Mg substrates, resulting in uncontrollable damage and delamination.^{[7]} Integrating organic anticorrosion coatings atop inorganic-coated Mg substrates may improve the interfacial adhesion, but this approach typically involves time-consuming and arduous operations.^{[8]} As a result, inorganic coatings are more desirable due to their stronger adhesion to Mg substrates, better mechanical durability, and facile preparation. Chromate conversion coating is the most frequently utilized inorganic anticorrosion coating but faces regulatory restrictions because of environmental concerns.^{[9]} Eco-and bio-friendly phosphate-based,^{[6a]} fluoride-based,^{[10]} or rare-earth-based^{[11]} conversion coatings have been developed but are vulnerable and intrinsically have low compactness due to their large particles. The large particles result from a mineralization process, where dissolved Mg ions are converted into inorganic minerals on Mg surfaces. They cannot densely pack on a substrate, causing mismatch-induced cracks as electrolyte pathways that enable corrosion. Moreover, conventional inorganic coatings lack antifouling properties and exhibit increased susceptibility to accelerated and complex bio-corrosion after implantation. Bio-corrosion involves a reciprocal deterioration of corrosion and fouling, where randomly distributed bio-foulants on the surface significantly hastening corrosion.^{[12]}

Therefore, there is a need in the art for an alternative coating for Mg-based surfaces which could benefit from anticorrosion function as well as antifouling function.

### SUMMARY OF THE INVENTION

In one aspect, described herein is a composite coating comprising an inorganic component comprising NaMgF₃ nanoparticles and a protein component comprising a water soluble, globular protein.

In some embodiments, the protein comprises albumin, such as bovine serum albumin. The protein component may be cross-linked.

In another aspect, described herein is a method of forming a composite coating on an Mg-based biomaterial, comprising the step of contacting the biomaterial with a solution of NaF and water soluble, globular protein.

In some embodiments, the protein comprises albumin, such as bovine serum albumin. The method may comprise the further step of cross-linking the protein after the coating is formed.

In another aspect, disclosed herein is a biomaterial comprising an Mg-based biomaterial coated with a composite coating as described herein. In some embodiments, the coated biomaterial is a surgical implant, such as a barrier membrane for guided bone regeneration (GBR) in dental regeneration surgery.

### BRIEF DESCRIPTION OF THE DRAWINGS

In the drawings, like elements are assigned like reference numerals. The drawings are not necessarily to scale, with the emphasis instead placed upon the principles of the present invention. Additionally, each of the embodiments depicted is but one of a number of possible arrangements utilizing the fundamental concepts of the present invention

FIG. 1. Illustration of Inorganic-protein (composite) coating strategy inspired by the biomineralization of tooth enamel formation. a, Tooth enamel formation based on protein-involved biomineralization and its ‘brick-and-mortar’ structure. b, Schematic diagram showing the comparison of energy change in the formation of conventional inorganic coatings and the new biomimetic composite coatings in this work. c, Schematic diagram showing the incorporation of BSA in and on the coatings achieves two synergistic functions: ‘mortar’ to seal residual cracks within the coatings to promote compactness, and mitigating biofouling issues.

FIG. 2. Preparation of composite coating. a, Facile one-pot preparation of NaMgF₃-BSA coating on Mg surfaces and corresponding XRD profiles. b, c, and d, FTIR spectra, XPS spectra, and O-PTIR images of inorganic-coated and composite-coated Mg surfaces. e, Density of the coatings. f and g, Morphology and thickness of inorganic and composite coatings. h, Fluorescent images of live/dead (green/red) staining of cells after culturing in extracts for for different time.

FIG. 3. Mechanical and anticorrosion properties of composite-coated Mg surfaces. a, Hardness of prepared coatings measured by nanoindentation. b, Friction coefficient and c, wear depth change during the friction test. d, Surface morphology and e, weight loss of pure Mg and coated-Mg immersed in PBS buffer for different durations. f, Nyquist and g, potentiodynamic polarization curves of pure Mg and prepared coated-Mg in PBS buffer. h, Weight loss of pure Mg and composite-coated Mg in different biofluids.

FIG. 4. Kinetics and thermodynamics in the formation of NaMgF₃-BSA composite. a, Reaction activation energy (ΔE) and Gibbs free energy (ΔG) change in the formation of NaMgF₃ and NaMgF₃-BSA. b, Mg ion concentration change in MgCl₂—NaF reactions at 30° C. with and without the presence of BSA molecules. c, Average diameter of NaMgF₃ and NaMgF₃-BSA particles. d, ITC curves showing the energy change of reactions. e, FTIR spectra showing the secondary structure of BSA protein. f, XRD profile for NaMgF₃ and NaMgF₃-BSA particles and (g) their characteristic peck position and crystallinity with different preparation duration.

FIG. 5. Antifouling performance of composite-coated Mg surface. a, Foulant distribution on inorganic and composite-coated Mg after immersed in blood and milk for 24 h and 48 h, measured by O-PTIR mapping. b, Monitoring dynamic adsorption of fouling on surfaces via QCM-D. c, Dynamic adsorption of blood and lysozyme on surfaces. d, Absorption/fouling amount of different bio-foulants on surfaces.

FIG. 6. Interfacial interactions between bio-foulants and inorganic/composite coatings measured by colloidal probe AFM. a, Schematic diagram showing experiment configuration in AFM force measurement and the interactions of typical foulants and functional groups with inorganic/composite coatings. b, Interfacial interactions (normalized force-distance, F/R-D, profiles) between Hb protein-coated surfaces and inorganic/composite coatings. c, Adhesion between different foulants and coatings.

FIG. 7. Application of composite coating on Mg-based barrier membrane. a, In-vitro Mg-Blood corrosion system. b, Tensile curve of pure Mg and coated Mg-membrane materials after testing for 8 days. c, Tensile strength of Mg-membrane materials with different testing duration. d, Fracture morphology of tested Mg-membrane materials.

FIG. 8. Morphology and surface composition of inorganic/composite-coated Mg sheets.

FIG. 9. Illustration of ‘Brick-Mortar’ structure of composite coating as well as STEM image and element distribution of developed composite coating.

FIG. 10. EDS spectra and element contents of inorganic/composite-coated Mg sheets.

FIG. 11. FTIR spectrum of native BSA protein.

FIG. 12. Static water contact angle (CA) of inorganic/composite-coated Mg sheets.

FIG. 13. Cell viability in the different extracts.

FIG. 14. Fluorescent images of live/dead (green/red) staining of cells after culturing in inorganic-coated Mg extracts for different time.

FIG. 15. Morphology of composite coating before and after test as well as the adhesion rate in the adhesion test of coatings to Mg substrates.

FIG. 16. The proposed models for the equivalent electrical circuits, where R_{S} represents solution resistance, R_{ct} is charge transfer resistance, Rₚ is the resistance of the porous Mg-Oxide surface formed in air, and R_{c} corresponds to the coating resistance.

FIG. 17. Bode plots of the pure Mg and coated-Mg surfaces fitted by proposed equivalent electrical circuit models.

FIG. 18. SEM images showing the morphology of pure Mg and composite-coated Mg during the corrosion in PBS buffer.

FIG. 19. EDS result of surface composition of pure Mg and composite-coated Mg after immersion in PBS for 20 min.

FIG. 20. EDS result of surface composition of pure Mg and composite-coated Mg after immersion in PBS for 16 days.

FIG. 21. FTIR result of corrosion product of pure Mg after immersion in PBS for 16 days.

FIG. 22. XRD result of corrosion product of pure Mg after immersion in PBS for 16 days.

FIG. 23. Potentiodynamic polarization curves of pure Mg and composite-coated Mg in PBS buffer for different duration.

FIG. 24. Weight loss of inorganic-coated Mg in different biofluids.

FIG. 25. Standard UV-Vis calibration curve of Mg-Calmagite solution with different Mg concentration and Mg concentration change during MgCl₂—NaF reaction

FIG. 26. Standard UV-Vis calibration curve of Mg(BSA)-Calmagite solution with different Mg concentration and Mg concentration change during MgCl₂—NaF-BSA reaction.

FIG. 27. XRD profiles of synthesized <Mg, Na, F> inorganics with different ratios of reactants.

FIG. 28. ITC measurement of the affinity between Mg ion and BSA protein.

FIG. 29. O-PTIR spectra of Milk and Blood (marked wavelength used as the characteristic peck for mapping).

FIG. 30. Histogram of typical foulant-surface interaction forces and their fitted Gaussian distribution.

FIG. 31. Morphology of pure Mg, inorganic, and inorganic-coated Mg-membrane materials tested in blood for different durations.

### DETAILED DESCRIPTION OF PREFERRED EMBODIMENTS

Aspects of the disclosed invention can provide an innovative coating approach to achieve dual protection for Mg-based biomaterials through using proteins to alter the reaction kinetics, resulting in coating design strategies for Mg-based metallic materials with desirable anticorrosion and antifouling performances.

As used herein, “Mg-based biomaterial” means a solid material which has or can be implanted or used in a living organism, and which comprises magnesium in either pure, alloyed or composite form.

In this specification, including the Figures, reference to “InorganicPro” is a reference to “Inorganic-Protein” which is an embodiment of the composite coating disclosed herein.

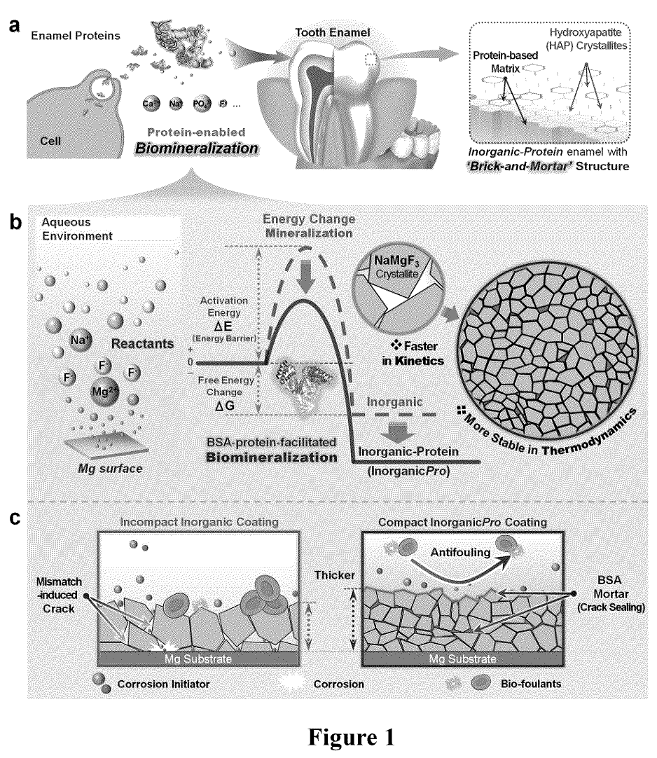

The concept for the composite coating strategy disclosed herein is inspired by the composite nature of tooth enamel. Tooth enamel is the hardest part of the human body, having a highly compact structure with fine hydroxyapatite (HAP) particles.^{[13]} It is an inorganic-organic composite, providing wear and corrosion protection. Tooth enamel formation is a typical mineralization process where enamel proteins, including amelogenin and non-amelogenin proteins, play a role in transforming Ca—Na—PO₄—F ions into hydroxyapatite (HAP) crystallites to construct enamel (FIG. 1a).^{[14]} Enamel proteins modulate the formation kinetics and thermodynamics of HAP via the protein-ion association, producing finer HAP crystallites to form densely packed enamel on the tooth (FIG. 1a).^{[16][17]}

Disclosed herein is a novel composite inorganic-protein coating, which is constructed in-situ on an Mg surface, which is preferably highly compact and which provides dual-protection. In one embodiment, the coating is produced through a protein-boosted reaction between sodium fluoride (NaF) and an Mg-based substrate. The association of Mg ions and the protein establishes a local hydrophobic domain that lowers the formation enthalpy of NaMgF₃ nanoparticles. The composite coating may be analogized to “brick and mortar” construction, where the nanoparticles function as ‘bricks’ and the protein acts as “mortar”.

In some embodiments, the protein is a water soluble globular protein, such as albumin. In preferred embodiments, the protein is a mammalian serum albumin such as natural or recombinant bovine serum albumin (BSA).

In some embodiments, the process produces finer nanoparticles which facilitates denser packing with fewer or smaller voids in coatings, thereby reinforcing mechanical durability.

The incorporation of the protein within and on the coatings plays at least two functions: (1) acting as a ‘mortar’ to seal residual cracks within coatings, thereby promoting coating compactness and increasing anticorrosion performance, and (2) mitigating fouling-accelerated bio-corrosion in complex biosystems by resisting bio-foulant attachments, including biofluids, proteins, and metabolites.

The introduced protein molecules enhance coating compactness by boosting Na—Mg—F biomineralization kinetics to generate more and finer NaMgF₃ nanoparticles. The finer inorganic nanoparticle ‘bricks’ densely pack on Mg surfaces, reducing mismatch-induced internal cracks and reinforcing the mechanical durability of coatings, significantly increasing anticorrosion performance.

Thus, in one aspect, disclosed is a composite coating comprising a water-soluble globular protein, such as bovine serum albumin (BSA), which upgrades general mineralization to biomineralization that converts Na and F ions into finer coating particles (FIG. 1b). As used herein, “biomineralization” means a biological process which forms mineral structures, such as by precipitation of inorganic compounds, in or on a living organism.

In some embodiments, such composite coating is prepared by immersing Mg-based surfaces into a solution with sodium fluoride (NaF) and BSA proteins. NaF will mineralize with the surface-released Mg ions, to form nanoparticles deposited on Mg substrates as an anticorrosion coating. BSA, an abundant protein in bovine plasma with high binding affinity to various ions,^{[15]} can affect the formation kinetics and thermodynamics of nanoparticles through biomineralization.

BSA proteins boost the kinetics of the Na—Mg—F mineralization, refine the deposited nanoparticles, and enhance the compactness of in-situ formed coatings-akin to the role of the enamel protein during tooth enamel formation. Meanwhile, the BSA macromolecules embedded inside composite coating act as ‘mortar’ to seal the diffusion pathways of corrosive mediums, further reinforcing the coatings.

BSA is a preferred protein as it is well recognized for its ability to inhibit biofouling and can anchor atop inorganic coatings, functioning as an antifouling layer.^{[18]} This anticorrosion and antifouling dual protection synergistically mitigates fouling-accelerated bio-corrosion in complex biological conditions, offering higher corrosion resistance and better antifouling performance than its conventional inorganic counterpart.

The underlying kinetics of BSA-involved biomineralization are believed to involve the association of BSA molecules with Mg ions to generate a locally hydrophobic domain, facilitating the decoordination between Mg ions and H₂O molecules. This, in turn, lowers the enthalpy of Na—Mg—F reaction (from ΔH₂=12.0 kJ mol⁻¹ to ΔH₁=7.9 kJ mol⁻¹) and kinetically boosts the nucleation of NaMgF₃ nanoparticles, reducing their average size from 652 nm to 503 nm. Moreover, the BSA macromolecules embedded inside composite coatings act as ‘mortar’ to seal mismatch-induced cracks between nanoparticles, forming a ‘brick-and-mortar’ structure of composite coating to further reinforce coating compactness and increase the interfacial electrochemical impedance fourfold.

The surface-anchored BSA molecules create an antifouling layer atop coatings to reduce adhesion between coatings and bio-foulants, enabling resistance to a broad spectrum of potential bio-foulants attachment, including metabolites, proteins, and biofluids. Synergizing with antifouling performance, the composite coating effectively mitigates the fouling-accelerated bio-corrosion and successfully secures exceptional anticorrosion properties in complex biological conditions.

Thus, in one aspect, described herein is a facile and effective preparation of a dual-protection coating on Mg-based biomaterials via employing proteins to alter the reaction kinetics, as well as incorporating the intrinsic antifouling functions of proteins into new coatings, which can be readily extended to engineer other inorganic coatings via inorganic-protein biomineralization, providing an innovative approach for developing novel coatings for various medical and non-medical applications, including drug mM delivery systems, hygiene and infection control, and fluid storage and transportation.

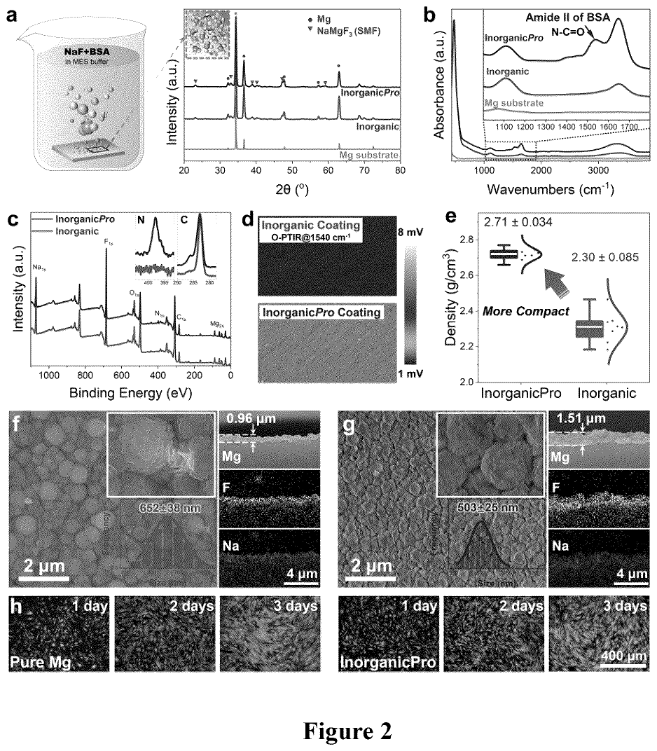

In some embodiments, the method of producing a coating comprises the step of contacting an Mg-based metallic surface with an aqueous solution of NaF and a protein. In one specific example, the composite coating can be facilely prepared by immersing a pure Mg surface into an aqueous solution containing NaF (500 mM) and BSA (10 mg mL⁻¹ in 10 mM MES buffer, pH=6) for 48 h at room temperature, as shown in FIG. 2a. Mg ions released from the solid surface interact with Na and F ions to form the NaMgF₃ coating, as indicated by X-ray diffraction (XRD) profiles.

In some embodiments, the coating may be stabilized by inducing cross-linking in the proteins. For example, to further stabilize the composite coating, it is dipped in a 10% glutaraldehyde solution for 10 min to cross-link BSA proteins in the coatings. In other examples, another cross-linker such as glyoxal or genipin may be used.

The more compact nature of composite coating provides mechanical durability and will also demonstrate anti-corrosion performance. The coating-substrate adhesion tests indicate that the developed coatings demonstrate good adhesion to the Mg substrate (FIG. 15).

Without restriction to a theory, it is believed the fine and densely packed nanoparticles of the composite coating provides anticorrosion performance. The finer and more densely packed nanoparticles can be the result of BSA molecules enzymatically boosting the nucleation of NaMgF₃ to generate more and smaller nanoparticles with lower reaction activation energy (ΔE).

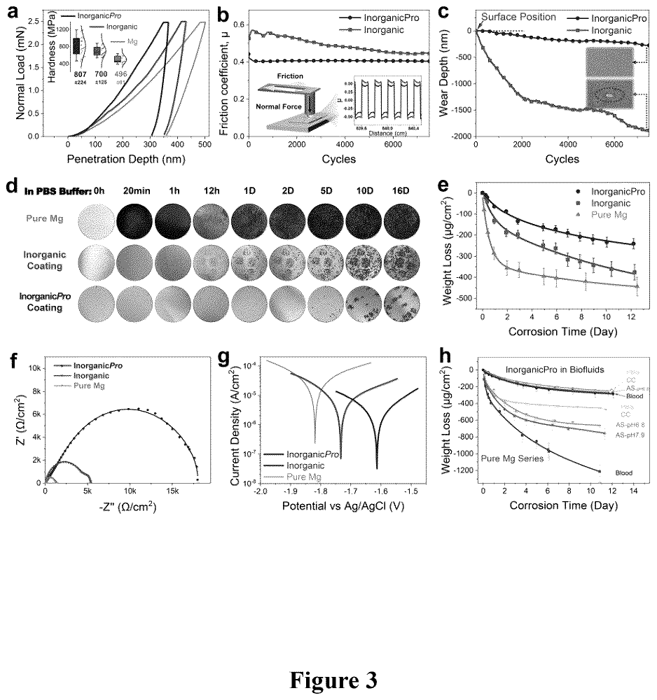

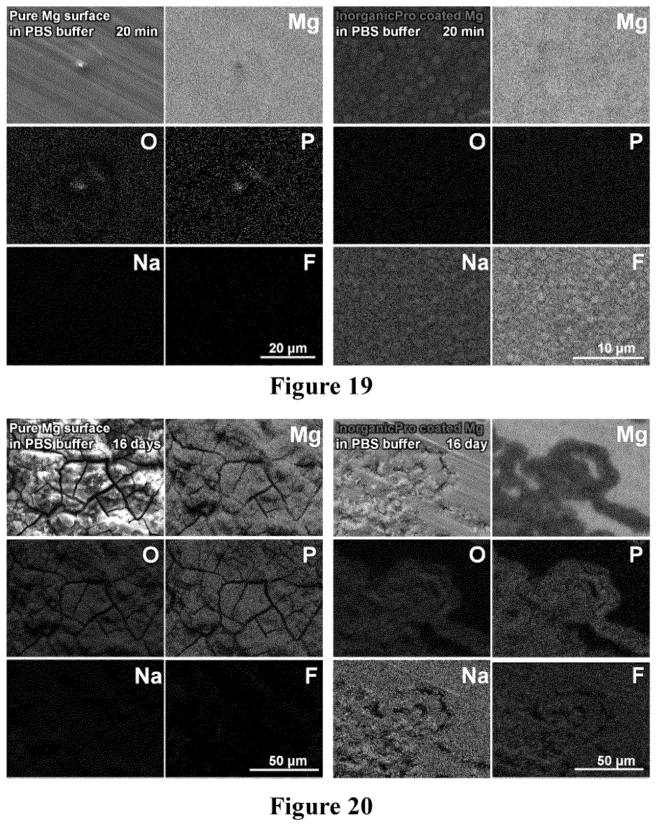

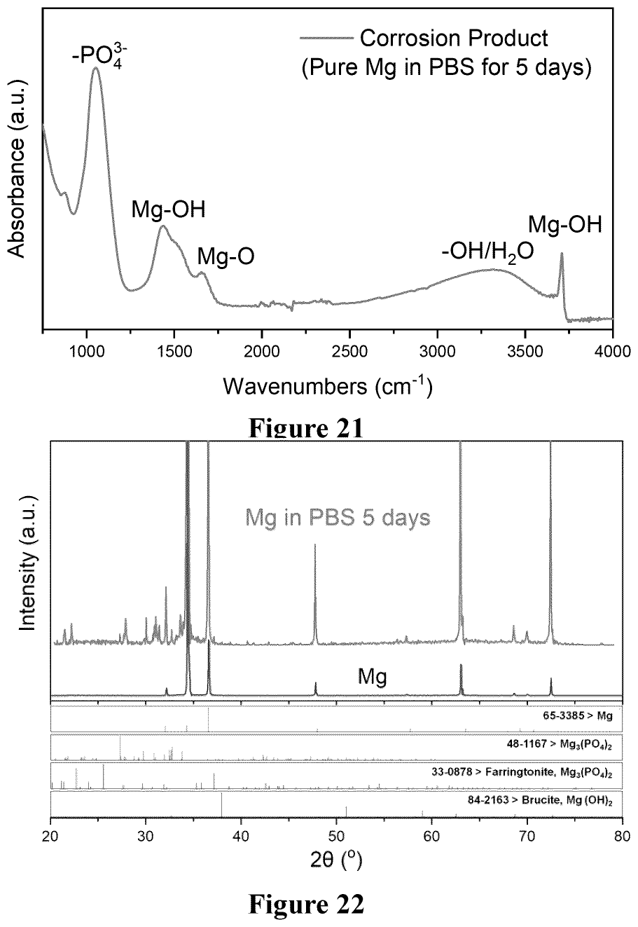

During a corrosion test in PBS buffer (FIG. 3d), dissolved Mg will convert to black corrosion products and accumulate on surfaces. The corrosion rate of Mg samples in PBS buffer is measured by the golden standard weight loss method, as summarized in FIG. 3e. An uncoated pure Mg surface rapidly develops a black and porous surface morphology with the fastest corrosion rate. The corrosion pits are observed on pure Mg surfaces after immersion in PBS for 20 min (FIG. 17) and corrosion products rapidly populate entire surfaces within 1 h of corrosion. The corrosion products are composed of high P and O elements (FIG. 19) and could be a mixture of Mg(OH)₂, Mg₃(PO₄)₂, and other products (FIGS. 21 and 22). The corrosion layer breaks into lots of separated fragments with obvious cracks on day 5 of corrosion. Consequently, the new Mg surface exposed in the crack area will experience further corrosion, producing a thicker corrosion layer. Although inorganic coating partially reduced this rapid corrosion, a lot of pitting corrosion can still be found on the bare inorganic-coated Mg surfaces.

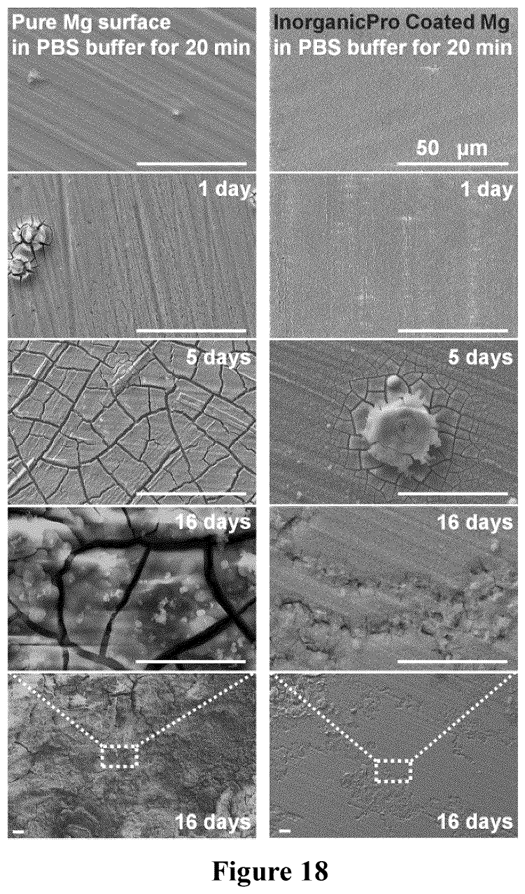

In contrast, the composite coating notably decreases the corrosion rate of Mg samples, maintaining a clean and shining surface after two days of corrosion. After 5 days of corrosion, composite-coated Mg undergoes small, localized corrosion (FIG. 18). This localized corrosion demonstrates a similar morphology to the corrosion pits of pure Mg surface and is composed of high P and O elements. In the following 10 days of corrosion, the corrosion area increases slowly compared with pure Mg and bare inorganic-coated Mg surfaces.

In an accelerated corrosion solution, abundant H₂ bubbles rapidly generate on the pure Mg surface, fewer bubbles on the inorganic-coated Mg surface, and no apparent bubbles are found on the composite-coated Mg surface, illustrating the exceptional corrosion resistance of composite coatings.

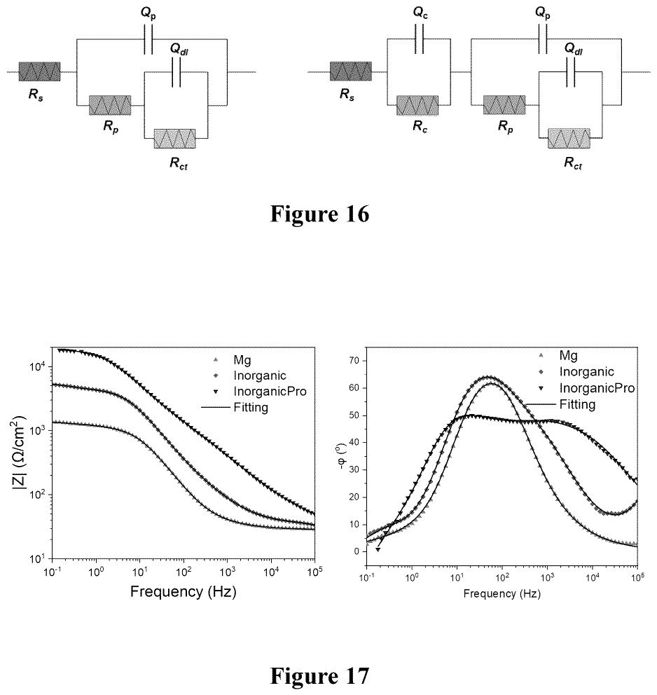

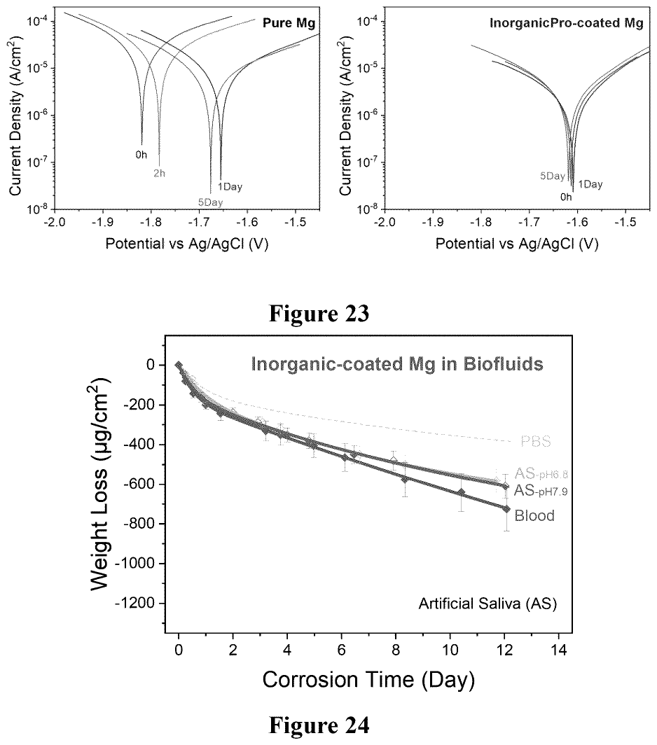

The anticorrosion performance of coatings was further investigated via electrochemical measurement in PBS buffer. Electrochemical impedance spectroscopy (EIS) data of different samples are analyzed using the equivalent circuit model in FIG. 16. As seen in the Nyquist plots (FIG. 3f), the composite-coated surface possesses the largest diameter of the capacitive semicircle compared with the pure Mg and bare inorganic-coated Mg surfaces. In the corresponding Bode diagram (FIG. 17), the composite coating features a low-frequency impedance modulus (|Z|0.1 Hz) of 1.81×10⁴ Ω cm⁻², nearly 4 times higher than that of the inorganic coating (5.21×10³ Ω cm⁻²) and 14 times of the pure Mg surfaces (1.36×10³ Ω cm⁻²). The potentiodynamic polarization results in FIG. 3g showcase that composite-coated surfaces demonstrate the highest corrosion potential (E_{corr}, −1.61 V) and lowest corrosion current density (i_{corr}, 2.46 μA cm⁻²). In comparison, the E_{corr} and i_{corr} of inorganic-coated are −1.73 V and 7.58 μA cm⁻², respectively. These findings indicate that the compact composite coating elevates the interfacial impedance, consequently slowing the corrosion of Mg in PBS buffer. In PBS buffer (FIG. 23), the corrosion potential of pure Mg surface first increases from −1.82 mV (0 h) to −1.65 mV (1 day) and then decreases to −1.68 mV (5 days). The increase in corrosion potential could be attributed to the formation of a P-containing corrosion layer on the Mg surface against the corrosion from PBS buffer. The corrosion layers break into small fragments after 1 day of corrosion due to the volume mismatch between corrosion products and Mg (FIG. 18), allowing new Mg surfaces to be exposed to PBS buffer for further corrosion, causing a decrease in overall corrosion potential. The corrosion current density continuously decreases during the 5 days of corrosion due to more corrosion products generated on the Mg surface blocking electron transfer.

In contrast, the corrosion potential of the composite-coated surface remains stably high and corrosion current density maintains a low value (FIG. 23), implying a lower corrosion rate. This low corrosion rate enables a longer time for P-containing corrosion layer formation in the small broken area of composite coating (FIG. 20) to mitigate further corrosion.

The corrosion behavior of Mg samples is also examined in different biofluids, including cell culture medium, artificial saliva, and blood, as shown in FIGS. 3h and 24. Compared with PBS buffer, various bio-foulants, such as proteins, metabolites, and cells, significantly accelerate the bio-corrosion process. It can be found that the corrosion ability varies among different biofluids, with blood>artificial saliva>cell culture medium≈PBS buffer. This ranking is attributed to the complexity of blood, which contains hundreds of proteins, metabolites, and various cells. In blood, after 10 days of corrosion, the weight loss of pure Mg in blood is 1176 μg cm⁻², which is almost 3 times that in PBS buffer (431 μg cm⁻²). For the inorganic-coated surface (FIG. 24), the weight loss is 635 μg cm⁻² in blood and twice as high as in PBS buffer (351 μg cm⁻²). Surprisingly, the composite-coated surface exhibits a weight loss of only 265 μg cm⁻² in blood, slightly higher in the PBS buffer (227 μg cm⁻²). This outstanding anticorrosion performance could be attributed to the more compact surface, mitigating the fouling-accelerated corrosion on the composite-coated Mg surface. These results highlight that the enhanced mechanical properties and superior anticorrosion performance of composite coating stem from the finer nanoparticles generated during BSA-involved biomineralization. Therefore, it is critical to understand the underlying mechanisms of particle refinement in the presence of BSA molecules.

In bio-corrosion, bio-fouling can accelerate surface corrosion, while corrosion-induced surface roughing leads to more bio-foulants adsorption, triggering a reciprocal deterioration, as shown in FIGS. 1c and 3h. Therefore, it is necessary to study the biofouling status on different coatings and investigate the role of surface-anchored BSA in antifouling performance.

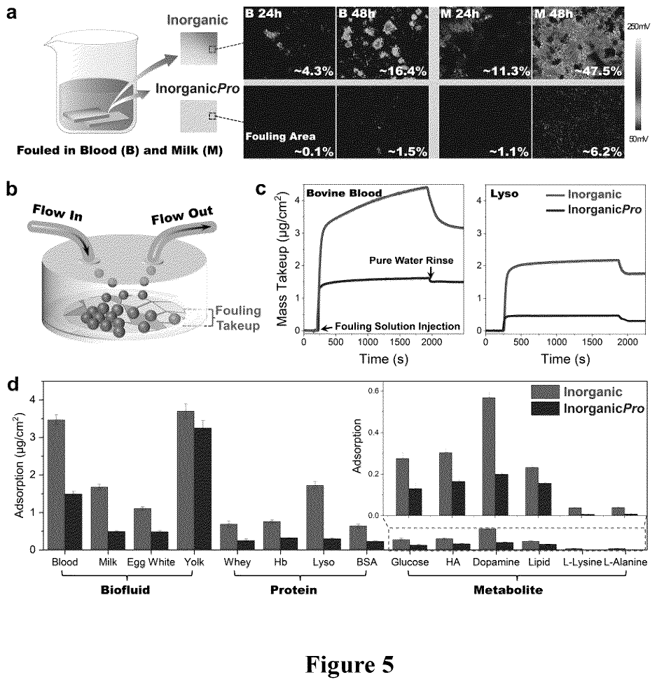

It is well acknowledged that the biofouling process is fundamentally governed by interfacial intermolecular interactions between surfaces and foulants, where attractive interactions would lead to more fouling. Such weak interfacial attraction can account for the high resistance to biofouling in FIG. 5. These weak interfacial interactions may stem from the interfacial hydration and steric repulsion from BSA molecules.

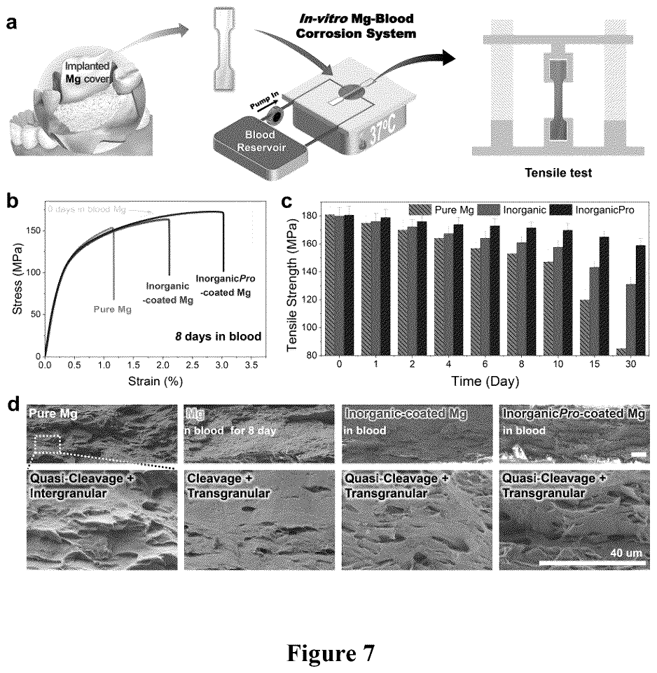

The composite coating with dual protection therefore can be used as a surgical implant, such as in implant dentistry, notably used as an Mg-based barrier membrane for guided bone regeneration (GBR) in dental regeneration surgery.^{[1e, 24]} GBR utilizes a barrier membrane to occlude soft-tissue cells, allowing slower-growing bone cells to repopulate the defect and regenerate bone, as shown in FIG. 7a. However, bio-corrosion poses a significant challenge to GBR, causing a severe deterioration in the mechanical properties of Mg-membranes, leading to the failure to encapsulate and support dental bone grafts. The composite coating disclosed herein can be used to protect the Mg-membrane against bio-corrosion.

### EXAMPLES

The following examples are intended to provide additional information about certain aspects or features of the disclosure above, and not to limit any claimed invention.

### Example 1. Materials

Bovine serum albumin (BSA), MES buffer, PBS buffer, Trichloro(octadecyl)silane (OTS), AgNO₃, NaCl, MgCl₂, CuCl₂, CrO₃ powders, glutaraldehyde, 16-mercaptohexadecanoic acid, 8-mercapto-1-octanol, (3-Aminopropyl) triethoxysilane (APTES), Reagent Alcohol (anhydrous), Calmagite, silica sphere powder, Whey, Hemoglobin (Hb), lysozyme (Lyso), Glucose, humic acid (HA), dopamine hydrochloride, L-lysine, artificial saliva, and L-Alanine were purchased from Sigma-Aldrich. NaF and RPMI 1640 medium were purchased from Fisher Scientific. Fetal bovine blood, milk, and Canola oil (lipid) were from the local grocery store. Ultrapure water in the experiments was from Milli-Q Advantage A10 (Millipore, USA). Mg sheet (0.2 mm in thickness) was purchased for Xintong Metals, China. The raw Mg sheet was annealed at 300° C. for 3 h before polishing and using in this work. Si sensors (QSX301) for QCM-D were from Biolon Scientific.

### Example 2. Measurement of the Mg Ion Concentration in Solution via UV-Vis Spectroscopy

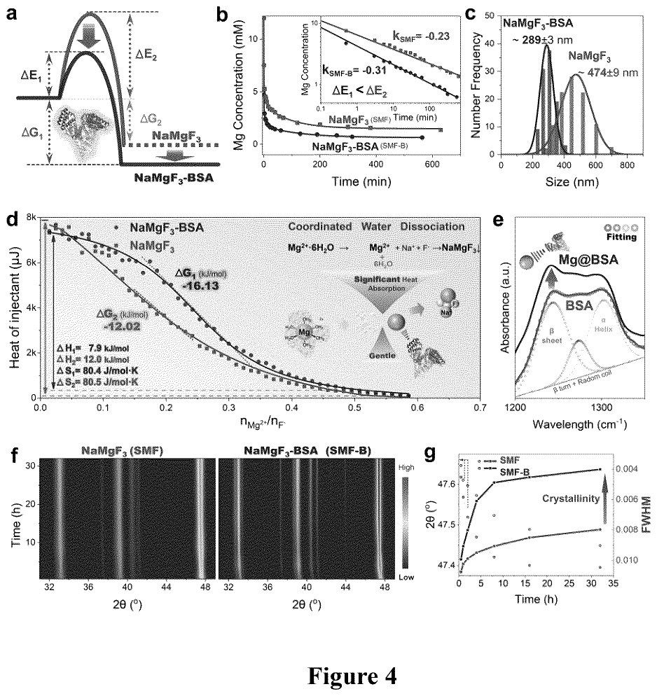

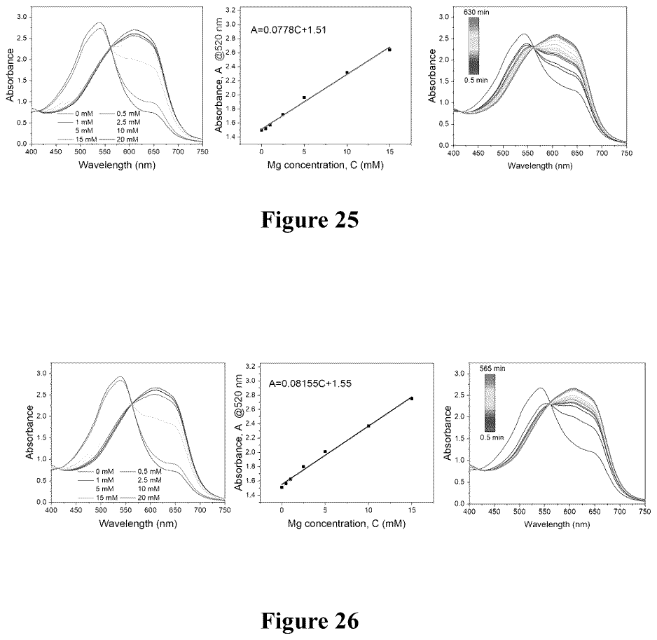

The reaction rate could be estimated by monitoring the Mg ion concentration change during the reaction between MgCl₂ (12 mM) and NaF (500 mM) at 30° C. The concentration of Mg ion could be measured by Calmagite solution (0.25 mM Calmagite in 3 mM Na₂B₄O₇ buffer, pH 9.25).^{[1]} 10 μL Mg ion solution was injected into 1 mL Calmagite solution and then, after 1.5 min, tested by UV-Vis spectrum on Evolution 300, Thermo Scientific, USA. The standard calibration curves and the Mg ion concentration change during the reaction with/without BSA are shown in FIGS. 25, 26, and FIG. 4b.

### Example 3. Isothermal Titration Calorimetry Measurement (ITC)

The ion-ion and ion-BSA binding was directly measured by ITC (NanoITC, TA, USA), in which the association constant (Kₐ), reaction stoichiometry (n), and the change in enthalpy (ΔH), entropy change (ΔS), and Gibbs free energy (ΔG) could be accurately determined.^{[2]} The affinity of Mg—Na—F with/without the presence of 10 mg mL⁻¹ BSA protein has been measured by ITC in FIG. 3d and S21, in which MgCl₂ salt solution (200 mM in 10 mM MES buffer, pH 6) was titrated into NaF solution (100 mM in 10 mM MES buffer, pH 6). In Mg-BSA affinity measurement, MgCl₂ salt solution (2 mM in 10 mM MES buffer, pH 6) was titrated into BSA solution (0.36 mM in 10 mM MES buffer, pH 6). In each measurement, 5 μL titrant solution was injected into 950 μL titrand solution with 50 injections. The reaction heat was corrected by subtracting the heat of titrant to buffer, buffer to titrand, and analyzed by the different models on the software NanoAnalyze by 1 independent onesite model. The enthalpy (ΔH) and reaction stoichiometry (n) for each reaction were listed in corresponding figures.

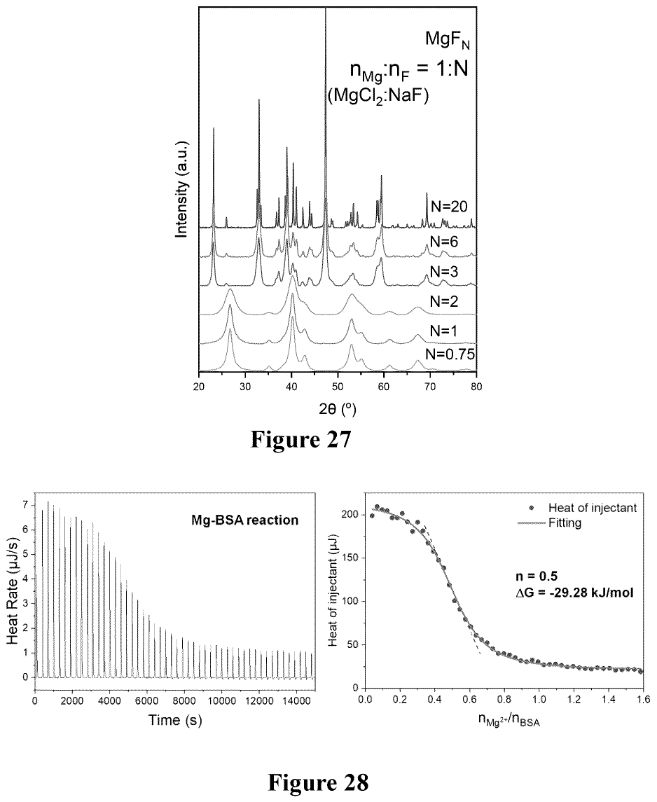

In a reaction kinetics test, the change in Mg ion concentration is monitored after injecting MgCl₂ (12 mM) into a NaF (500 mM) solution with and without the presence of BSA molecules at 30° C. (FIG. 4b, S18, and S19) .^{[19]} It is evident that Mg ions rapidly react in the initial stage and reach a final plateau after 100 min. Na—Mg—F reaction is faster in the presence of BSA, as shown in the insert subfigure of FIG. 4b. The faster reaction suggests the activation energy with the presence of BSA is lower than that without BSA (ΔE₁<ΔE₂).^{[20]} With boosted reaction kinetics, more NaMgF₃ nanoparticles nucleate within 1 h, and consequently, the average diameter of nanoparticles with the assistance of BSA is reduced to 289 nm, much smaller than that without BSA (474 nm in FIG. 4c). The thermodynamic parameters of the Na—Mg—F reaction are measured by the isothermal titration assays (ITC, FIG. 4d). It is considered that Na—Mg—F formation is a two-step reaction: in the first step, six coordinated water molecules dissociate from Mg ions with significant heat absorption to produce bare Mg²⁺ ions;^{[21]} in the second step, the Mg²⁺ reacts with F⁻ and Na⁺, forming NaMgF₃ nanocrystals (FIG. 27). Due to a large amount of heat absorbed during the Mg—H₂O decoordination, the overall reaction is endothermic with an enthalpy change (ΔH₂) of 12.0 kJ mol⁻¹, as displayed in FIG. 4d. When BSA molecules are present in Na—Mg—F solution, the Mg ions will react with BSA before with F⁻ ions, as Gibbs free energy of BSA-Mg reaction (ΔG_{BSA-Mg}=−29.28 kJ mol⁻¹, FIG. 28) is much lower than that of Na—Mg—F reaction (ΔG₂=−12.02 kJ mol⁻¹). Once Mg and BSA bond, the more hydrophobic β sheet structure will form in the BSA-Mg association (FIG. 4e). This association creating a local hydrophobic domain will facilitate the decoordination of Mg−H₂O and reduce the reaction enthalpy from ΔH₂=12.0 kJ mol⁻¹ to ΔH₁=7.9 kJ mol⁻¹. By reducing the reaction barrier posed by H₂O molecules, the reaction between Mg and F will become more kinetically favorable and faster. The BSA-boosted Na—Mg—F reaction also facilitates the formation of NaMgF₃ crystals. The higher crystallinity of NaMgF₃ nanoparticles (FIGS. 4f and 4g) and lower Gibbs free energy (FIG. 4d) in the presence of BSA suggests that NaMgF₃-BSA is more stable. The Mg-BSA association enables Mg ions as active anchor sites for BSA molecules to glue the inorganic NaMgF₃ particles, strengthening the hardness and contributing to a better wear resistance of composite coating, as indicated in FIG. 3a-3c. In the novel coating design, involving BSA in biomineralization not only reinforces the coating compactness to improve the anticorrosion performance but also grants coatings antifouling properties to mitigate the fouling-accelerated bio-corrosion, as suggested in FIG. 3h.

### Example 4. Measurement of Mechanical Properties and Density of Coatings

The mechanical properties of the coatings, such as the hardness and friction behavior, are essential for the long-term operation of implants, which are measured on the NHT nano indenter NTR³ nano tribometer (FIG. 3a-c, Anton Paar, Austria). In the nanoindentation test, the Berkovich indenter measured the hardness with 2.5 mN of normal loading force. At least 30 different points were tested and the averaged curves were plotted in FIG. 3a. The nanoindentation curves were analyzed by using Oliver-Pharr theory to calculate the surface hardness. In the reciprocating ball-on-disk friction test, a nylon ball with a diameter of 2 mm as a counter-facing ball was contacted with the coating in a NaF (100 mM) aqueous solution with 30 mN of loading force. The ball reciprocally slid on the surfaces with a velocity of 1 mm s⁻¹ and sliding amplitude 1 mm for different cycles. Typical shear force and friction coefficient versus sliding distance/cycle were displayed in FIG. 3b, and wear depth during the friction was shown in FIG. 3c.

The density of the coatings was determined by the weight and volume change before and after coating removal by a CrO₃ solution for 10 min, following the American Society of Testing Materials (ASTM) standard method (ASTM B659-90 R2014). Specifically, the weight and volume of the coated sample were measured using an analytic balance and Archimedes' principle, respectively. After removing the coatings, the weight and volume of the sample were re-measured. The change in weight and volume of the sample was then considered as the weight and volume of the coating. The density of the coatings could be calculated by their weight and volume accordingly.

The coating adhesion test (ASTM-D-3359-09) is employed to assess the adhesion of coatings to substrates. Specifically, cuts are made on the surface of dry samples, and then a brush is used to remove any detached flakes. Place tape over the grid smoothly and firmly with a finger to ensure good contact with the coating. After 90 s of application, remove the tape by seizing the free end and rapidly (not jerked) back upon itself at as close to an angle of 180° as possible. Inspect the grid area for removal of coating from the substrate, and finally rate the adhesion in accordance with the scale illustrated in ASTM-D-3359-09.

Nanoindentation in FIG. 3a indicates that composite exhibits higher hardness (807 MPa) than a conventional inorganic coating (700 MPa), likely attributed to its higher density. A nylon ball that represents the average mechanical properties of contact surfaces in dental applications is employed for friction with the coatings. It is found that the finer and evenly distributed nanoparticles in composite coating make the surface smoother with a lower friction coefficient (FIG. 3b). Consequently, the wear depth of composite coating is much lower and there is no friction-induced surface damage after long-term friction (FIG. 3c).

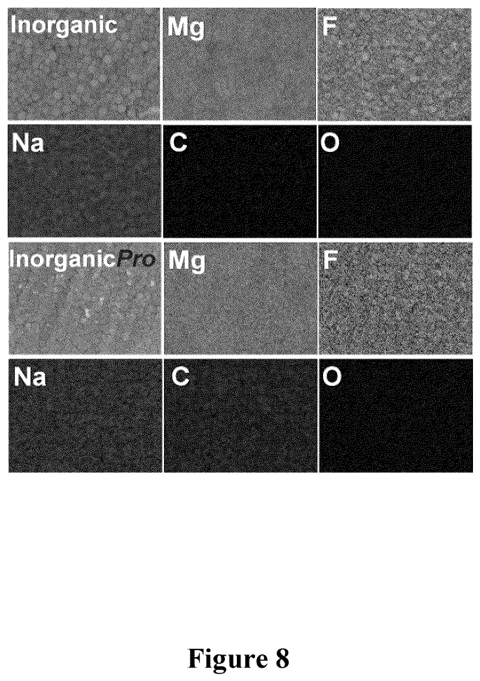

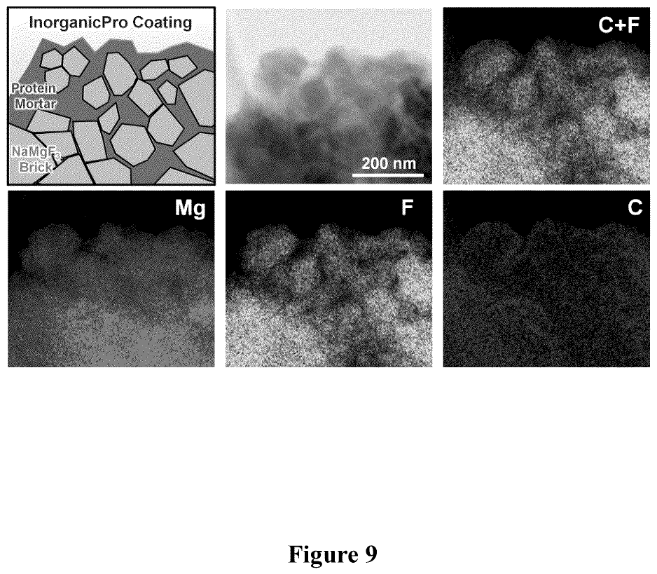

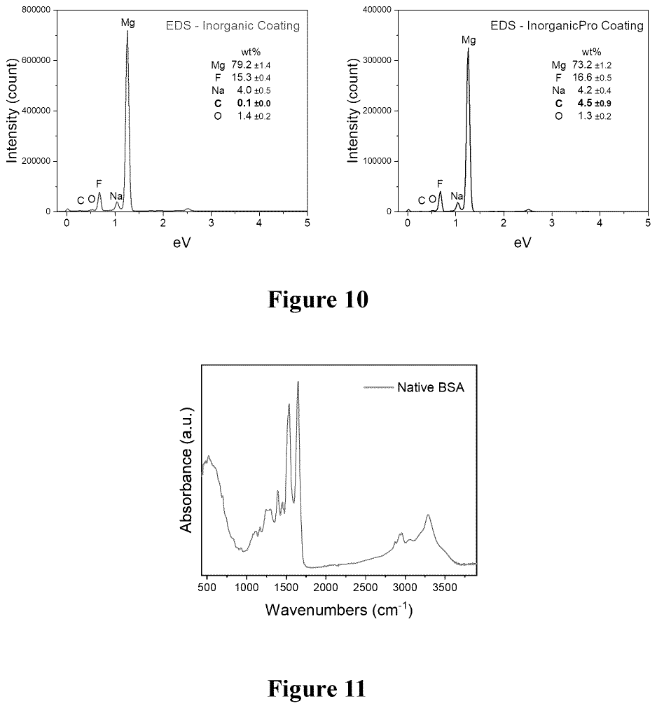

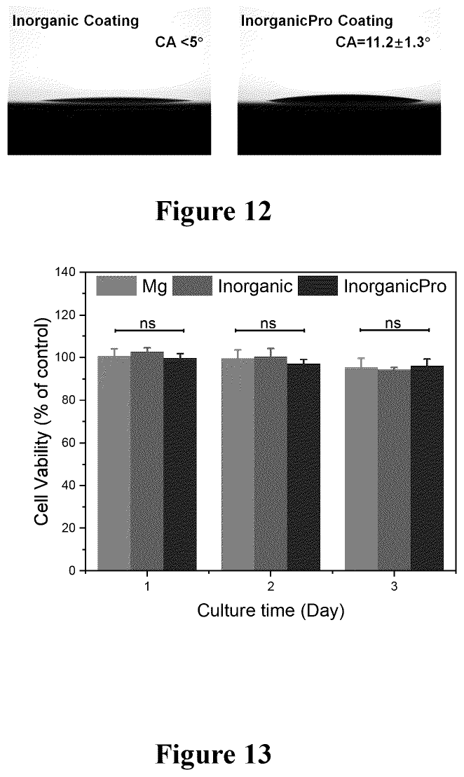

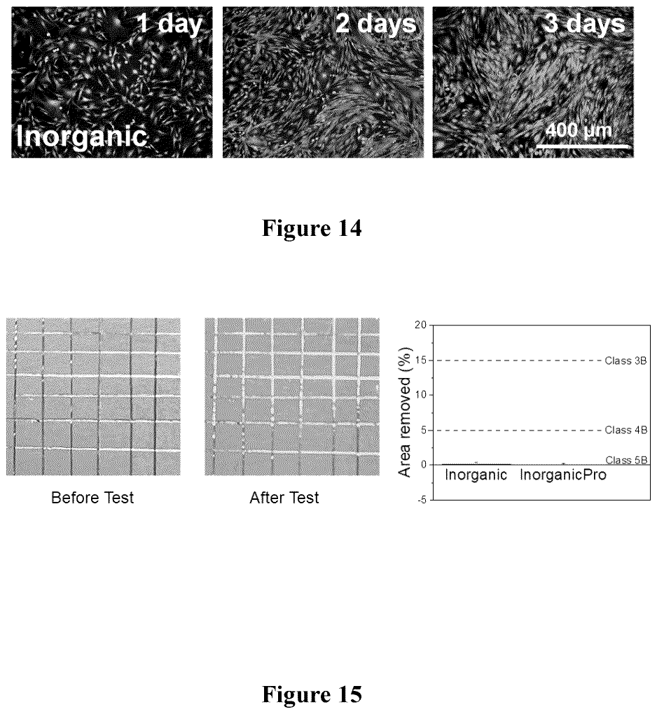

The characteristic peak of BSA at 1540 cm⁻¹ in Fourier transform infrared (FTIR, FIG. 1b and S4) spectra of the coatings indicate the presence of BSA in the composite coating. A high carbon content (5%) observed in energy dispersive spectroscopy (EDS, FIGS. 8 and 10) suggests the abundant embedding of BSA molecules within the composite coating. A distinct N peak in X-ray photoelectron (XPS, FIG. 1c) spectra, notable intensity of optical photothermal infrared (O-PTIR, FIG. 1d) image at 1540 cm⁻¹, and higher contact angle (FIG. 12) of composite coating show that lots of BSA molecules anchor on the surface of composite coating. The scanning transmission electron microscopy (STEM) and corresponding EDS images of composite coating in FIG. 9 clearly demonstrate its ‘brick-mortar’ structure. The experimentally observed distribution of BSA aligns with the illustration in FIG. 1c, demonstrating that BSA both embeds within and anchors on the surface of the coating. The morphology of coatings is displayed in FIGS. 1f and 1g. The average size of nanoparticles in composite coating is 503 nm in diameter, finer than that in bare inorganic coating deposited in the absence of BSA (652 nm). The composite coating is also thicker than that of its inorganic counterpart. To quantify the compactness of the prepared coatings, we measure the density of the coatings based on an American Society of Testing Materials (ASTM) standard method (ASTM B659-90 R2014). The estimated density of the composite coating is 2.71 g cm⁻³ and denser than that of the inorganic coating of 2.30 g cm⁻³ (FIG. 2e). As the density of pure solidified NaMgF₃ crystal is 3.03 g cm⁻³, the calculated compactness of inorganic coating is ~75.9%, with the volume of mismatch-induced cracks inside the inorganic coating being ~24.09 vol %. In contrast, the compactness of the composite coating is as high as 89.4%, with only 10.56 vol % of cracks inside the coating, suggesting that finer nanoparticles reduce cracks in coatings by 56.16 vol %, contributing to a denser and more compact composite coating, as proposed in FIG. 1. This denser and more compact coating can provide improved mechanical and anticorrosion performance to protect Mg surfaces.

### Example 5. Cytotoxicity

Human gingival fibroblasts (HGF, ScienCell, American) were used to evaluate cell biocompatibility in vitro and incubated in complete medium including Dulbecco's modified Eagle's medium (DMEM), 10% fetal bovine serum (FBS), and 100 IU mL⁻¹ penicillin and 100 IU mL⁻¹ streptomycin (Gibco, American). Before the cell test, all samples (pure Mg, inorganic-coated Mg, and composite-coated Mg) were irradiated with ultraviolet light for two hours for sterilization. The sterilized samples were immersed in the medium at 37° C. for 24 h to obtain the extracts and the ratio of the surface area of the sample to the volume of the medium was 3 cm² mL⁻¹. Then, the HGF cells were seeded in a 96-well plate with a density of 2,000 cells per well and cultured in a humidified incubator with 5% CO₂ at 37° C. for 24 h. The culture medium was then changed to the sample extracts and cultured for 1, 2, and 3 days with the complete medium as the control group, respectively. The 10 μL CCK-8 (Cell Counting Kit-8, Bestbio, China) solution was added to each well and further cultured in a cell incubator for 2 h. Then the absorbance at 450 nm was determined by a microplate spectrophotometer (SpectraMax paradigm, Molecular Devices, USA).

The HGF cells were seeded in 96-well plate with a density of 3,000 cells per well and cultured in a humidified incubator at 37° C. for 24 h. The culture medium was then changed to the sample extracts and cultured for 1, 2, and 3 days with the complete medium as the control group, respectively. Then, the samples were washed with phosphate buffered solution (PBS, pH 7.4) and stained with Calcein-AM/EthD-1 (Invitrogen, American) for 30 min at 37° C. After staining, the samples were rinsed with PBS and the live/dead cells on the sample surface were observed with an inverted fluorescence microscope (Olympus, Japan).

The biocompatibility of coated Mg samples is evaluated by CCK-8 assay (FIG. 13) through culturing human gingival fibroblasts (HGF) in sample extracts for 1, 2, and 3 days. It can be found that the Mg-based biomaterials, including pure Mg and coated Mg samples, exhibit excellent biocompatibility with cell viability higher than 96% after 3 days of cell proliferation (FIG. 12). The fluorescent images of live/dead staining of cells in FIGS. 2h and 12 show the increase in the number of living cells during the culture. This test demonstrates the good biocompatibility of the coated-Mg samples in this work.

### Example 6. Corrosion Test and Weight Loss Measurements

A series of pure Mg and coated Mg was corroded in different bioflouids, including 1×PBS buffer cell culture-medium (RPMI 1640 Medium) supplemented with 10% FBS (Fetal Bovine Serum), artificial saliva at pH 6.8 and 7.9, and 20% diluted blood for different durations. The lost Mg will be dissolved into the corrosion solution or form corrosion products on the surface. Such weight loss of Mg was measured by the golden standard method—using CrO₃ (200 g L⁻¹)+AgNO₃ (10 g L⁻¹) to remove the corrosion products on Mg surfaces. Specifically, corroded Mg was immersed in CrO₃ solution for 20 min. Then, the clean Mg was rinsed with water and dried before measuring the weight change. The weight loss will be normalized by surface area for different samples. An accelerated corrosion solution (5 wt % NaCl+2 mM CuCl₂) was used to examine the corrosion rate of different Mg surfaces.

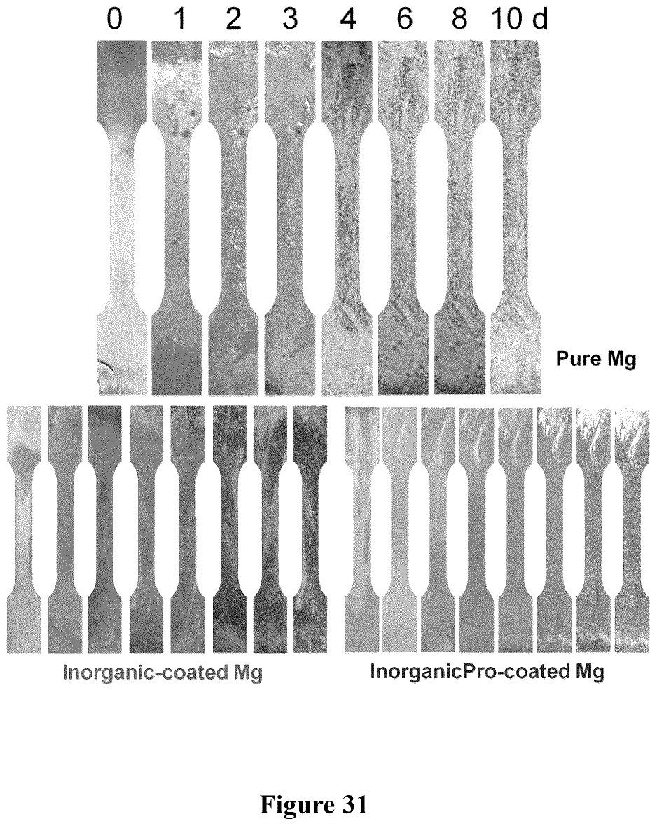

We use the most corrosive biofluids tested in this work, blood, to challenge the protective performance of the coatings in an in vitro bio-corrosion test. Specifically, All the Mg samples are incubated in an in-vitro Mg-Blood corrosion system that simulates the post-implantation conditions. After 1 day, a porous corrosion layer appears on the pure Mg surface (FIG. 31). After 2 days, pitting corrosion is found on the inorganic-coated surface. After 4 days of corrosion, some sparsely distributed corrosion is found on the composite surface; in contrast, continuous and thick corrosion products already cover the entire pure Mg surface. The mechanical properties of Mg samples are tested and summarized in FIGS. 7b and 7c. After 30 days of corrosion, the tensile strength of pure Mg is only 85.1 MPa, 47.1% of the original one, and that of bare inorganic-coated Mg is 131.2 MPa, 72.7% of the original value. In contrast, the tensile strength of composite-coated Mg is twice as high as that of pure Mg, demonstrating a high strength of 159.2 MPa, 88.5% of the original value. The fracture morphology in FIG. 7d shows that the pure Mg without corrosion is a quasi-cleavage and intergranular fracture and then transfers to cleavage and transgranular fracture after corrosion, which may result from the released hydrogen diffusion inside the grains of Mg samples. The composite-coated sample exhibits hydrogen embrittlement but demonstrates quasi-cleavage patterns similar to Mg without corrosion. The dual protection coating reduces the corrosion rate in blood and helps keep the desirable mechanical properties of Mg-based biomaterials, benefiting their potential applications in clinical utility.

### Example 7. In Vitro Biofouling Test

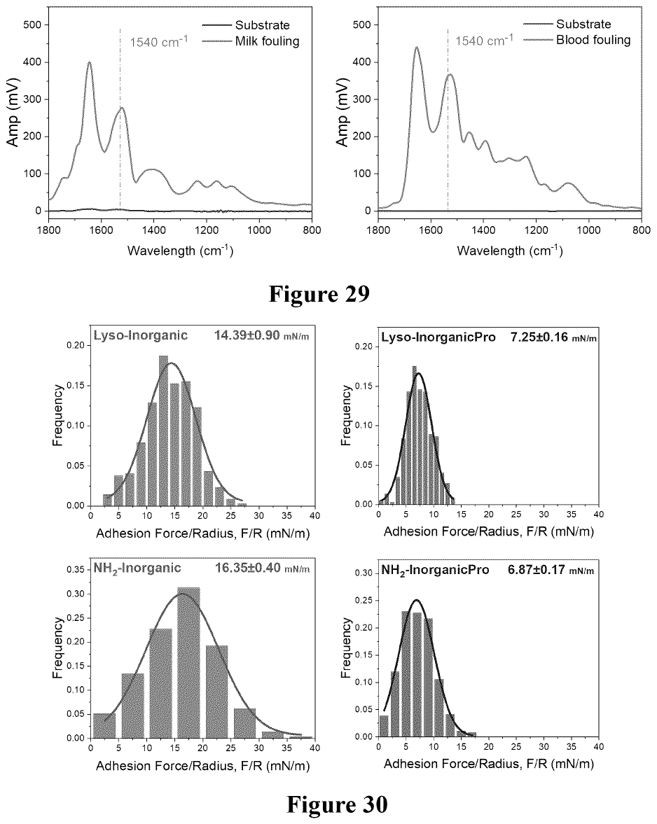

The inorganic/composite-coated Mg were incubated in bovine blood and milk for 24 h and 48 h, respectively, to evaluate the fouling resistance of coatings. After fouling, the surfaces were copiously rinsed with ultrapure water before characterization. Bio-foulants on the surfaces in the tests have been characterized using O-PTIR, a non-contact submicron visible probe infrared spectroscopy (mIRage, Photothermal Spectroscopy Corp, CA). In O-PTIR characterization, a tunable pulsed mid-infrared (IR) laser induces photothermal effects onto a sample surface, which are measured using a scattered visible probe laser to focus on the sample. The reflection IR spectra recorded by O-PTIR can be correlated to ATR-FTIR spectra. The O-PTIR spectra of bio-foulants in blood and milk (FIG. 29) showed their corresponding characteristic peaks. Then, using IR at 1540 cm⁻¹ (blood as an example) to scan the sample surfaces with the resolution of 500 nm to map the milk foulants distributed on the sample surfaces (FIG. 5a). The O-PTIR mapping overcomes the limitation of EDS characterization, which cannot identify organic groups with the same composition, and the spatial resolution limitation of traditional FTIR microscopy (10-20 μm). The coverage of foulants on surfaces was measured on two samples with 3 different areas. The results from the biofouling test demonstrated the excellent antifouling properties of composite coating.

FIG. 5a displays the bio-foulant distribution on inorganic and composite coatings after incubation in blood and milk for different durations. The O-PTIR images reveal that after fouling 48 h in blood, around 16.4% of the surface area on the inorganic coating is occupied by foulants. In contrast, only 1.5% of the surface area is contaminated on the composite coating, indicating around 10 times better fouling resistance. Similarly, the composite coating also exhibits outstanding fouling resistance in milk solution, with only 6.2% surface fouled after 48 h. To further examine the antifouling capability of composite coatings against typical bio-foulants in biosystems, such as biofluids, proteins, and metabolites, the dynamic adsorption of biomolecules on coatings is directly monitored in real-time using a quartz crystal microbalance with dissipation monitoring (QCM-D, FIG. 5b). As shown in FIG. 5c, a strong mass takeup is observed upon the introduction of blood into the QCM-D chamber, indicating the adsorption of foulants from blood on both inorganic and composite surfaces. After 30 min, ultrapure water was introduced into the chamber to remove the loosely bonded foulants on coatings, decreasing mass takeup. It is found that the fouling from the blood or Lyso protein solution on the composite coating is considerably lower than that on the bare inorganic coating. Composite coating also exhibited high-efficiency fouling resistance to a broad spectrum of bio-foulants, as summarized in FIG. 5d. For instance, the remnant bovine blood on the composite coating after rinsing with water is ~1.49 μg cm⁻², while those on the inorganic coating are as high as 3.47 μg cm⁻². The remnant Lysozyme protein on the composite coating is only ~0.30 μg cm⁻², while those on the inorganic coating are as high as 1.72 μg cm⁻². This superior antifouling property most likely originates from the surface-anchor BSA that provides interfacial hydration and steric repulsion to hinder the initial attachment of foulants on surfaces.^{[18a, 22]} Such interfacial interactions between the coatings and typical foulants could be quantified by direct interaction force measurements.

### Example 8. Quartz Crystal Microbalance With Dissipation Monitoring (QCM-D) Test

20% dilution of biofluids and protein (5 mg mL⁻¹) and other water-soluble metabolites were used in this test. For the lipid (Canola oil, a highly viscous oil employed as a model lipid), it was dissolved in ethanol with a concentration of 20 mg mL⁻¹ and dispersed via ultrasonic for 30 min, and then the lipid/ethanol mixture was dispersed in water via a homogenizer (T18 digital ULTRA TURRAX, IKA, Germany, speed 20,000 rpm, 15 min). The inorganic and composite surfaces were obtained by the dip-drying method, in which inorganic/composite particles were dispersed in ultrapure water and then dipped on the APTES-functionalized Si sensors and dried for 1 h. The coated sensor was rinsed with ultrapure water to remove the loosely bonded particles before QCM-D tests on Q-Sense E4 (Biolin Scientific, Finland). The data analysis follows the extended viscoelastic model on QTools software (Biolin Scientific, Finland). The adsorption value was averaged by 3 measurements.

### Example 9. Interfacial Interaction Forces Measurements by Colloidal Probe Atomic Force Microscopy (AFM)

AFM nanomechanical study is a direct and quantitative analysis technique for interaction forces between two components^{[3]}. This work measured the interaction forces between the foulant-coated AFM probe and inorganic/composite coatings^{[23]}. Protein (Whey, Hb, Lyso)-coated AFM probe was prepared via the dip-coating method, in which a silica colloidal AFM probe was firstly prepared by gluing a silica microsphere with a diameter of ~5 μm onto a tipless cantilever using epoxy glue and then was immersed in protein solution (0.5 mg mL⁻¹ protein) and incubated for 1 h, followed by thorough rinsing with Milli-Q water and drying by nitrogen. —NH₂ and —CH₃ functional group coated AFM probe was prepared via immersing silica probe in APTES (20 mM in anhydrous reagent alcohol) and OTS (20 mM in anhydrous reagent alcohol), respectively, for 2 h, followed by thorough rinsing with reagent alcohol and drying by nitrogen. —COOH and —OH coated AFM probe was prepared via immersing gold-coated probe in 16-mercaptohexadecanoic acid (10 mM in anhydrous reagent alcohol) and 8-mercapto-1-octanol (10 mM in anhydrous reagent alcohol) for 12 h, followed by thorough rinsing with reagent alcohol and drying by nitrogen.

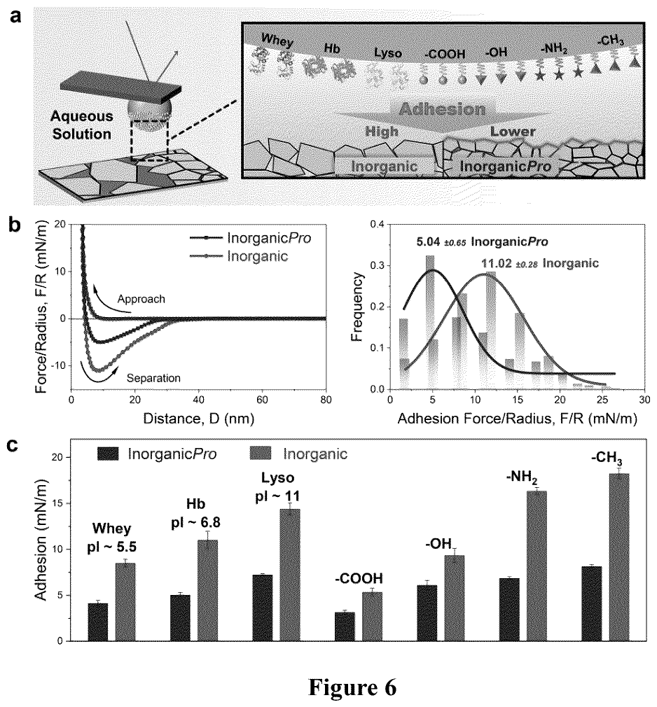

AFM force measurements between foulant-coated AFM probe and inorganic/composite coatings were carried on Bruker ICON AFM in 1×PBS buffer at pH 7.4. To ensure the accuracy of force measurements, force mapping was performed on various coatings in an area of 10×10 μm² to acquire a two-dimensional array of force-separation profiles with 15×15 points in at least three different regions of the coatings and at least two independently prepared samples of the same batch. The Gaussian method statistically analyzed the measured interfacial interaction forces, as shown in FIG. 6b and FIG. 30.

The experimental setup of AFM force measurements is illustrated in FIG. 6a, where adhesion between protective coatings and surfaces covered with Whey, Hb, and Lyso proteins as well as —COOH, —OH, —NH₂, and —CH₃ functional groups are measured in PBS buffer. The normalized force-distance (F/R-D) profiles in FIG. 5b show that the adhesion (F_{ad}/R) of Hb protein to inorganic coating is 11.02 mN m⁻¹, twice as strong as that to composite coating at 5.04 mM m⁻¹, suggesting that surface-anchored BSA weakens the interfacial interactions between bio-foulants and composite surfaces. Likewise, the adhesion of a series of bio-foulants to composite coatings is only 40-50% of their adhesion to inorganic coatings (FIG. 6c). For example, the adhesion of Lyso protein to composite coating is 50% of that to inorganic coating, and the adhesion of amino-coated surface to composite coating is 42% of that to inorganic coating (FIG. 30).

### Example 10. In Vitro Mg-Blood Corrosion Test

The Mg-based devices will be under bio-corrosion after implantation, where the blood continuously fouls and corrodes the surface of Mg-implants, resulting in rapid degradation of surface integrity and mechanical properties. The surface morphology and mechanical strength of Mg-implants during bio-corrosion were characterized by an in-vitro Mg-Blood corrosion test, as shown in FIG. 7a. The pure Mg barrier membrane materials were incubated in blood at 37° C. for several days. After bio-corrosion, the tensile strength of Mg-membrane materials was measured and their fracture morphology was analyzed by scanning electron microscopy (SEM), as indicated in FIG. 7.

### Example 11. Other Characterizations

Glancing Incidence X-ray Diffraction (GI-XRD) of coatings on Mg substrates was conducted on Rigaku XRD Ultima IV, Rigaku, Japan, with a glancing angle of 0.5°. Power XRD measurements were performed on D8 Discover, Bruker, Germany. X-ray photoelectron spectroscopy (XPS) was from Kratos AXIS Ultra, UK. The binding energy was calibrated by C₁ₛ peak at 284.6 eV. Attenuated Total Reflection-Fourier-transform infrared spectroscopy (FTIR) was performed on Nicolet iS50, Thermo Scientific, USA. Static water contact angle (CA) in the air of various substrates was measured with a 3 μL of ultrapure water droplet. Scanning electron microscopy (SEM) and energy dispersive spectrometer (EDS) characterizations were performed on Sigma GEMINI FE-SEM, ZESSI, Germany. Scanning transmission electron microscopy-energy dispersive spectrometer (STEM-EDS) characterization was performed on JEM-ARM200CF, JEOL, Japan. Particle size measurements were carried out on a Zetasizer Nano ZSP system, Malvern Panalytical, UK. Electrochemical Impedance Spectroscopy (EIS) and potentiodynamic polarization tests were performed on the electrochemical workstation, CH Instruments, USA. Tensile tests were conducted on AGS-X universal tensile testing machine, Shimadzu, Japan, with the tensile speed 1 mm min⁻¹.

### Interpretation

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” or “~” can refer to a variation of ±5%, ±10%, ±20%, or ±25% of the value specified. For example, “about 50” percent can in some embodiments carry a variation from 45 to 55 percent. For integer ranges, the term “about” or “~” can include one or two integers greater than and/or less than a recited integer at each end of the range. Unless indicated otherwise herein, the term “about” or “~” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

### REFERENCES

The following references are indicative of the level of skill in the art, and are incorporated herein by reference in their entirety, where permitted.
[1] a) J. L. Rutkowski, Vol. 47, The American Academy of Implant Dentistry, 2021; b) S. Pal, S. Pal, Design of Artificial Human Joints & Organs 2014, 377; c) V. P. Mantripragada, B. Lecka-Czernik, N. A. Ebraheim, A. C. Jayasuriya, Journal of biomedical materials research Part A 2013, 101, 3349; d) A. Chaya, S. Yoshizawa, K. Verdelis, N. Myers, B. J. Costello, D.-T. Chou, S. Pal, S. Maiti, P. N. Kumta, C. Sfeir, Acta biomaterialia 2015, 18, 262; e) Z.-Y. Yan, J.-H. Zhu, G.-Q. Liu, Z.-C. Liu, C.-B. Guo, N.-H. Cui, J.-M. Han, Bioinorganic Chemistry and Applications 2022, 2022.
[2] J. L. Wang, J. K. Xu, C. Hopkins, D. H. Chow, L. Qin, Adv Sci (Weinh) 2020, 7, 1902443.
[3] a) Y. Wang, B. Liu, X. Zhao, X. Zhang, Y. Miao, N. Yang, B. Yang, L. Zhang, W. Kuang, J. Li, Nature communications 2018, 9, 4058; b) L.-Y. Li, Z. Yang, X.-X. Pan, B.-X. Feng, R. Yue, B. Yu, Y.-F. Zheng, J.-Y. Tan, G.-Y. Yuan, J. Pei, Advanced Functional Materials 2022, 32, 2205634; c) W. Xu, N. Birbilis, G. Sha, Y. Wang, J. E. Daniels, Y. Xiao, M. Ferry, Nature materials 2015, 14, 1229; d) M. Esmaily, J. Svensson, S. Fajardo, N. Birbilis, G. Frankel, S. Virtanen, R. Arrabal, S. Thomas, L. Johansson, Progress in Materials Science 2017, 89, 92; e) D. Zang, R. Zhu, W. Zhang, X. Yu, L. Lin, X. Guo, M. Liu, L. Jiang, Advanced Functional Materials 2017, 27, 1605446; f) M. Cihova, P. Schmutz, R. Schäublin, J. F. Löffler, Advanced Materials 2019, 31, 1903080.
[4] a)F. Witte, Acta biomaterialia 2010, 6, 1680; b) Y. Zheng, X. Gu, F. Witte, Materials Science and Engineering: R: Reports 2014, 77, 1; c) G. Uppal, A. Thakur, A. Chauhan, S. Bala, Journal of Magnesium and Alloys 2022, 10, 356.
[5] P. Tong, Y. Sheng, R. Hou, M. Iqbal, L. Chen, J. Li, Smart Materials in Medicine 2022, 3, 104.
[6] a) H. Hornberger, S. Virtanen, A. R. Boccaccini, Acta biomaterialia 2012, 8, 2442; b) S. Amukarimi, M. Mozafari, MedComm 2021, 2, 123.
[7] a) T. Yimyai, D. Crespy, M. Rohwerder, Advanced Materials 2023, 2300101; b) R. G. Hu, S. Zhang, J.-F. Bu, C.-J. Lin, G.-L. Song, Progress in Organic Coatings 2012, 73, 129.
[8] M. Toorani, M. Aliofkhazraei, M. Mahdavian, R. Naderi, Corrosion Science 2021, 178, 109065.
[9] a) S. Liao, B. Yu, X. Zhang, X. Lu, P. Zhou, C. Zhang, X. Chen, T. Zhang, F. Wang, Journal of Magnesium and Alloys 2021, 9, 505; b) P. L. Hagans, C. Haas, ASM Handbook 1994, 5, 405.
[10] a)Y. Qiu, X. Tu, X. Lu, J. Yang, Corrosion Science 2022, 199, 110177; b )H. Zheng, Z. Li, M. Chen, C. You, J. Wang, Advanced Engineering Materials 2023, 25, 2201364; c) J. Lou, Y. Sun, Y. Chen, R. Zan, H. Peng, S. Yang, X. Kang, Z. Peng, W. Wang, X. Zhang, Surface and Coatings Technology 2021, 422.
[11] V. S. Saji, Journal of Materials Research and Technology 2019, 8, 5012.
[12] a)T. Yimyai, R. Thiramanas, T. Phakkeeree, S. Iamsaard, D. Crespy, Advanced Functional Materials 2021, 31; b) L. Zhao, Q. Liu, R. Gao, J. Wang, W. Yang, L. Liu, Corrosion Science 2014, 80, 177.
[13] H. Zhao, S. Liu, Y. Wei, Y. Yue, M. Gao, Y. Li, X. Zeng, X. Deng, N. A. Kotov, L. Guo, Science 2022, 375, 551.
[14] a) S. Ali, I. Farooq, Protein and Peptide Letters 2019, 26, 880; b) A. G. Fincham, J. P. Simmer, presented at Ciba Foundation Symposium 205—Dental Enamel: Dental Enamel: Ciba Foundation Symposium 205, 2007; c) A. Veis, Science 2005, 307, 1419.
[15] Z. Zhao, M. Pan, W. Yang, C. Huang, C. Qiao, H. Yang, J. Wang, X. Wang, J. Liu, H. Zeng, Journal of Colloid and Interface Science 2023, 650, 1525.
[16] a) J. H. Harding, D. M. Duffy, M. L. Sushko, P. M. Rodger, D. Quigley, J. A. Elliott, Chemical reviews 2008, 108, 4823; b) S. Mann, 2001; c) A. Navrotsky, Proceedings of the National Academy of Sciences 2004, 101, 12096; d) S. R. Qiu, C. A. Orme, Chemical reviews 2008, 108, 4784; e) S. Shi, W. Fan, R. Tao, H. Xu, Y. Lu, F. Han, S. Yang, X. Zhou, Z. Zhou, F. Wan, ACS Biomaterials Science & Engineering 2020, 7, 133; f) Y. Song, S. Zhang, J. Li, C. Zhao, X. Zhang, Acta Biomaterialia 2010, 6, 1736; g) Y. Su, D. Li, Y. Su, C. Lu, L. Niu, J. Lian, G. Li, ACS Biomaterials Science & Engineering 2016, 2, 818.
[17] Z. Yu, X. Li, X. Li, B. Zheng, D. Li, D. Xu, F. Wang, Advanced Functional Materials 2023.
[18] a) Z. Zhao, M. Pan, C. Qiao, L. Xiang, X. Liu, W. Yang, X. Z. Chen, H. Zeng, Adv Mater 2022, e2208824; b) X. Hu, J. Tian, C. Li, H. Su, R. Qin, Y. Wang, X. Cao, P. Yang, Adv Mater 2020, 32, e2000128; c)J. Sabate Del Rio, O. Y. F. Henry, P. Jolly, D. E. Ingber, Nat Nanotechnol 2019, 14, 1143.
[19] a) M. Ryan, H. Barbour, Annals of clinical biochemistry 1998, 35, 449; b) M. C. Yappert, D. B. DuPre, Journal of Chemical Education 1997, 74, 1422.
[20] a)E. D. Dill, J. C. Folmer, J. D. Martin, Chemistry of Materials 2013, 25, 3941; b) A. Elabbar, Materials Chemistry and Physics 2019, 238, 121896.
[21] a) O. Allnér, L. Nilsson, A. Villa, Journal of chemical theory and computation 2012, 8, 1493; b) L. Bellarosa, S. Calero, N. López, Physical Chemistry Chemical Physics 2012, 14, 7240; c) T. Dudev, J. Cowan, C. Lim, Journal of the American Chemical Society 1999, 121, 7665.
[22] J. N. Israelachvili, Intermolecular and surface forces, Academic press, 2011.
[23] a) L. Xie, X. Cui, J. Liu, Q. Lu, J. Huang, X. Mao, D. Yang, J. Tan, H. Zhang, H. Zeng, ACS Appl Mater Interfaces 2021, 13, 6941; b) W. Yang, M. Pan, J. Zhang, L. Zhang, F. Lin, X. Liu, C. Huang, X. Z. Chen, J. Wang, B. Yan, H. Zeng, Advanced Functional Materials 2021; c) D. Yang, X. Peng, Q. Peng, T. Wang, C. Qiao, Z. Zhao, L. Gong, Y. Liu, H. Zhang, H. Zeng, Engineering 2022.
[24] Y. Guo, Y. Yu, L. Han, S. Ma, J. Zhao, H. Chen, Z. Yang, F. Zhang, Y. Xia, Y. Zhou, Materials Science and Engineering: C 2019, 100, 226.

### Additional References

[1] a) M. Ryan, H. Barbour, Annals of clinical biochemistry 1998, 35, 449;
b) M. C. Yappert, D. B. DuPre, Journal of Chemical Education 1997, 74, 1422.
[2] a) A. Singh, P. Datta, L. M. Pandey, Int J Biol Macromol 2017, 97, 662;
b) J. T. Duskey, F. da Ros, I. Ottonelli, B. Zambelli, M. A. Vandelli, G. Tosi, B. Ruozi, Molecules 2020, 25;
c) W. R. Archer, M. D. Schulz, Soft Matter 2020, 16, 8760.
[3] a) L. Xie, X. Cui, J. Liu, Q. Lu, J. Huang, X. Mao, D. Yang, J. Tan, H. Zhang, H. Zeng, ACS Appl Mater Interfaces 2021, 13, 6941;
b) L. Xie, F. Liu, J. Liu, H. Zeng, ACS Appl Mater Interfaces 2020, 12, 58360.

1. A composite coating comprising an inorganic component comprising NaMgF₃ nanoparticles and a protein component comprising a water soluble, globular protein.

2. The composite coating of claim 1 wherein the protein comprises albumin.

3. The composite coating of claim 2 wherein the albumin comprises bovine serum albumin.

4. The composite coating of claim 1 wherein the protein component is cross-linked.

5. A method of forming a composite coating on an Mg-based biomaterial, comprising the step of contacting the biomaterial with a solution of NaF and water soluble, globular protein.

6. The method of claim 5 wherein the protein comprises albumin.

7. The method of claim 6 wherein the albumin comprises bovine serum albumin.

8. The method of claim 4 comprising the further step of cross-linking the protein after the coating is formed.

9. A biomaterial comprising an Mg-based biomaterial coated with a composite coating of claim 1.

10. The biomaterial of claim 9 which is a surgical implant.

11. The biomaterial of claim 10 which is a barrier membrane for guided bone regeneration (GBR) in dental regeneration surgery.
